(19) United States
(12) Patent Application Publication
(10) Pub. No.: US 2007/0177017 A1
(43) Pub. Date: Aug. 2, 2007

(54) Stress detection device and methods of use thereof

(76) Inventors: Bobby Kyle, Danielsville, GA (US); Gabe Abreo, Kansas City, MO (US); Vardis Benson, Braselton, GA (US); Brad E. Kays, Dacula, GA (US)

Correspondence Address: GARDNER GROFF SANTOS & GREENWALD, P.C., 2018 POWERS FERRY ROAD, SUITE 800, ATLANTA, GA, 30339, US

(21) Appl. No.: 11/345,095

(22) Filed: Feb. 1, 2006

(51) Int. Cl. H04N 5/225; A61B 6/00

(52) U.S. Cl. 348/207.99; 600/473

(57) ABSTRACT

Described herein are stress detection devices and methods of use thereof.

### BACKGROUND

The detection of stress has numerous applications in the security field. For example, in airports, it would be desirable to measure or detect stress of individuals that have been singled out and will be interviewed. Military applications include interviewing suspected terrorists in fixed facilities and in remote locations. In other instances, stress can be an indication of untruthfulness. Thus, for example, the detection of stress can be useful during police interrogations as an indicator of untruthfulness. In addition, the detection of stress can be used by parole boards to help determine stress levels when addressing certain topics that would be sensitive to the reason for the incarceration of the individual. The detection of stress in the corporate world can be useful when interviewing personal for sensitive job positions in high security applications.

In general, the detection of stress in a subject involves invasive techniques, where a device needs to be physically attached to the subject. With security applications, invasive techniques are impractical and offer little value. Thus, it would be desirable to detect stress levels in a subject without the subject knowing he is being examined. One approach is to measure the skin temperature of the subject. U.S. Pat. No. 5,771,261 to Anbar describes how dynamic area telethermometry (DAT) can be used to detect changes in skin temperature and skin perfusion and subsequently correlate the changes to mental stress. Thus, the use of infrared cameras to measure changes in a subject's skin temperature during police interrogations can be an indication of whether or not the subject is being honest when responding to questions.

Although the use of infrared cameras provides useful information about the stress level of a subject, the results in the absence of other data can be misleading. For example, changes in temperature in a police interrogation room may result in an increase in skin temperature. Further, prolonged interrogations can also provide a steady increase in skin temperature. Thus, what is needed is a device that can simultaneously measure or detect a plurality of stress indicators in a subject. For example, by simultaneously measuring or detecting changes in several features of the subject, it is possible to more reliably determine whether or not the subject is being truthful. Described herein are stress detection devices and methods of using thereof that overcome the limitations of previous stress detection devices.

### SUMMARY

Described herein are stress detection devices and methods of using such devices. The advantages of the materials, methods, and articles described herein will be set forth in part in the description which follows, or may be learned by practice of the aspects described below. The advantages described below will be realized and attained by means of the elements and combinations particularly pointed out in the appended claims. It is to be understood that both the foregoing general description and the following detailed description are exemplary and explanatory only and are not restrictive.

### BRIEF DESCRIPTION OF THE DRAWINGS

The accompanying drawings, which are incorporated in and constitute a part of this specification, illustrate several aspects described below. It will be appreciated that these drawings depict only typical embodiments of the materials, articles, and methods described herein and are therefore not to be considered limiting of their scope.

FIGS. 1A and 1B shows a schematic of one aspect of the stress detection device.

FIGS. 2A and 2B shows a schematic of another aspect of the stress detection device.

FIG. 3 shows an application of a stress detection device in an interrogation room.

FIG. 4 shows a durable laptop with an imaging box mounted on the laptop.

### DETAILED DESCRIPTION

Before the present devices and methods are disclosed and described, it is to be understood that the aspects described below are not limited to specific compounds, synthetic methods, or uses as such may, of course, vary. It is also to be understood that the terminology used herein is for the purpose of describing particular aspects only and is not intended to be limiting.

Throughout this specification, unless the context requires otherwise, the word “comprise,” or variations such as “comprises” or “comprising,” will be understood to imply the inclusion of a stated integer or step or group of integers or steps but not the exclusion of any other integer or step or group of integers or steps.

It must be noted that, as used in the specification and the appended claims, the singular forms “a,” “an” and “the” include plural referents unless the context clearly dictates otherwise. Thus, for example, reference to “a pharmaceutical carrier” includes mixtures of two or more such carriers, and the like.

“Optional” or “optionally” means that the subsequently described event or circumstance can or cannot occur, and that the description includes instances where the event or circumstance occurs and instances where it does not.

Different components can be used to produce the devices described herein. It is understood that when combinations, subsets, interactions, groups, etc. of the components are disclosed that while specific reference of each various individual and collective combinations and permutation of these components may not be explicitly disclosed, each is specifically contemplated and described herein. For example, if a number of components are disclosed and discussed, each and every combination and permutation of the component are specifically contemplated unless specifically indicated to the contrary. This concept applies to all aspects of this disclosure including, but not limited to, steps in methods of using the disclosed devices. Thus, if there are a variety of additional steps that can be performed it is understood that each of these additional steps can be performed with any specific embodiment or combination of embodiments of the disclosed methods, and that each such combination is specifically contemplated and should be considered disclosed.

Described herein is a stress detection device, comprising

(a) a thermal imager for detecting changes in skin temperature in a subject during the questioning of the subject, wherein the thermal imager produces a thermal analog representation, a thermal digital representation, or a combination thereof of the subject;

(b) a video camera for observing the subject during the questioning of the subject, wherein the video camera produces a video analog representation, a video digital representation, or a combination thereof of the subject;

(c) a means for interleaving or de-interleaving multiple representations from (a) and (b);

(d) a means for providing a reference point between audio output data and the representations from (a) and (b); and

(e) a display device for displaying the representations and the audio output data.

Each component of the device will be discussed below, followed by a discussion of how each component is connected to produce the stress detection device.

The thermal imager for detecting changes in skin temperature of a subject can be an infrared camera that can continuously monitor the modulation (i.e., increase or decrease) of skin temperature. In certain aspects, the infrared camera can detect changes in skin temperature where the skin of the subject is exposed. In one aspect, the changes in skin temperature are detected at the face, including the ears, cheeks, neck, and forehead. The mechanism of which infrared cameras can measure skin temperature caused by stress is discussed in U.S. Pat. No. 5,771,261 to Anbar. It is contemplated that the infrared camera can detect all ranges of infrared energy emitted from the subject, including near infrared (0.7 μm to 1.4 μm), short-wave infrared (1.4 μm to 3 μm), mid-wave infrared (3 μm to 8 μm), long-wave infrared (8 μm to 15 μm), far infrared (15 μm to 1,000 μm), and any combination thereof. It is also contemplated that two or more thermal imagers can be used. For example, one infrared camera can be used to measure long-wave infrared energy while a second infrared camera can be used to measure mid-wave infrared energy. The term “detect” as used herein involves qualitative changes in skin temperature. Quantitative changes in skin temperature can be measured, which will be discussed below. The infrared camera produces analog and/or digital representations of the subject, which is subsequently converted to video output data. This will be discussed in greater detail below. Any infrared camera can be used herein. In one aspect, infrared cameras such as, for example, K6800, K6700 and K6900 manufactured by ISG can be used herein

In certain aspects, it may be desirable to quantify the change in skin temperature as a measure of stress in a subject. In one aspect, the device further comprises a means for detecting statistically significant changes in periodic modulation of skin temperature of the subject. In one aspect, a cluster analysis can be performed to measure statistically significant changes in skin temperature. The cluster analysis disclosed in U.S. Pat. No. 5,771,261 to Anbar, which is incorporated by reference, can be used herein. In cluster analysis, which is well known in the art, each frequency-amplitude pair of a fast Fourier transform (FFT) spectrum generated by the thermal imager (e.g., infrared camera) is represented by a dot on a plane that is described by amplitude versus frequency coordinates. Different FFT spectrum generated by the thermal imager are represented by a different clusters on the same plane. The two clusters may partially overlap, and a mathematical procedure, well known in the art, can determine the level of significance of the difference between the two spectra, represented by the extent of overlap of the two clusters. A common measure of the difference between two clusters is the calculated probability (p) that the two clusters compared are actually members of a single cluster, i.e., that statistically there is no difference between those two clusters. It is generally accepted that if p is smaller than 0.05 (i.e., that there is less than a 5% chance that the two clusters are identical) the difference between the two clusters is statistically significant. In another aspect, the stress detection device does not measure quantitatively changes in skin temperature such as, for example, statistically significant changes in skin temperature described above.

One or more video cameras can be used in the device to provide video of the subject during questioning. In one aspect, the video camera comprises at least one CCD camera. In another aspect, the video camera comprises two CCD cameras. The number and types of cameras that can be used will vary depending upon the application and location of the device (portable vs. fixed systems). In certain aspects, the video camera that is used can cover a narrow field of vision that is aligned at the same angle as that of the thermal imager (e.g., infrared camera). In this aspect, the video camera is focused on the face of the subject. In other aspects, the video camera can be a wide field of vision camera that can be used to film the entire body of the subject or the upper torso of the subject. It is contemplated that one or more narrow and wide field of view cameras can be used and aligned at various angles with respect to the subject to provide as much visual information as needed about the subject during questioning. The video camera(s) produce(s) video analog and/or digital representations of the subject that can then be subsequently converted to video output data.

The thermal imager and video camera produce analog and/or digital representations of the subject that can be converted to video output data (i.e., visual data). This can be accomplished by a means for interleaving or de-interleaving the representations produced by the thermal imager and video camera to produce video output data, wherein the video output data can be displayed. The term “interleave” is defined herein as the combination of two or more data streams (e.g., analog and/or digital representations) in a time-division sequence arranged according to a specified set of rules such as alphabetically, numerically, or chronologically resulting in a single data stream. “De-interleaving” refers to the segmentation and separation of a data stream according to a specified set of rules such as alphabetically, numerically, or chronologically resulting in multiple data streams.

In one aspect, the means for interleaving or de-interleaving the representations produced by the thermal imager and video camera to produce video output data comprises a multiplexer. A multiplexer is a device that takes multiple digital streams and combines them into one data stream. Thus, it is contemplated that data produced by the thermal imager and the video camera can be converted (i.e., interleaved) to a single signal with the use of a multiplexer, where the signal can ultimately be displayed. It is also contemplated that audio data can also be interleaved with the video data produced by the thermal imager and video camera to produce a single signal.

In another aspect, the multiplexer can be computer software imbedded in a personal computer or laptop. The software can perform numerous functions including, but not limited to, (1) a way for visually displaying each video signal that is configurable to the users required functionality, which includes but is not limited to, single video display mode, dual video display mode, triple video display mode, quad video display mode, quad video display mode with audio, or any combination of the above; (2) a way of recording any/all input signals to an on-board or removable storage device in a format that can be easily distributed; (3) a way of visually displaying each audio signal that is configurable to the users required functionality; and/or (4) a way of playing back any recording of audio/video stored by the user.

In another aspect, the means for interleaving or de-interleaving the thermal analog and/or digital representations produced by the thermal imager and the video analog and/or digital representations from the video camera to produce video output data comprises a quad splitter. The quad splitter permits the display of multiple video streams produced by the thermal imager and the video camera. The quad splitter also permits the display of audio data (e.g., changes in amplitude and frequency), which can be displayed with the video data. Thus, the quad splitter can produce multiple screens or view points derived from multiple sources of audio and video data.

Commercially-available quad splitters are self-enclosed pieces of hardware that provide multiplexing and display mode selections for either black & white or color video signals. The units are available from several manufacturers with the RQS-10B as an example from SPECO Technologies. The quad splitter can be connected to any video recording device either analog or digital. In another aspect, the quad splitter can be an integrated quad computer video card. In this aspect, the computer video card mechanically connects multiple video and audio streams to a PC sub-system. The computer video card also interleaves multiple video and audio signals into a single data stream such that it can be placed on a communication bus for processing. Any of the video cards manufactured by ILDVR USA can be used herein. The use of video cards as the quad splitter permits the manufacture and use of portable stress detection devices. In other aspects, a multiplexer can be used in combination with a quad splitter to interleave multiple video and audio streams.

The stress detection device also has a means for providing a reference point between audio output data and the video output data. In one aspect, audio data from a subject can be generated with the use of a microphone connected to a computer comprising software for analyzing changes in voice frequency, voice amplitude, or a combination thereof. The software produces a graphic representation of changes in frequency and/or amplitude of the subject's voice during questioning. In one aspect, Adobe Audition can be used to record the audio stream and generate the graphic representation. It is contemplated that the microphone can be wireless or connected to the computer by cable(s). The importance of establishing the reference point between audio output data and the video output data will be discussed in detail below.

In certain aspects, it may be desirable to detect changes in audio stress level of the subject during the questioning of the subject. For example, software can be optionally employed to detect and measure stress levels in a subject's voice. Computer software is readily available to one of ordinary skill in the art for detecting audio stress levels of the subject. In one aspect, TVSA3 stress analysis software can be used.

In other aspects, the device has a means for recording audio, video, and thermal data generated by the device. The means for recording the data can also store data or transfer the data to other data storage devices. The means for recording the data can be a master storage device that can be sterilized by removing all of the data after the audio data has been transferred to another data storage device, thus keeping the data contained and secure. In one aspect, the means for recording the audio data is software imbedded in a computer (e.g., PC or laptop).

The stress detection devices described herein generate video and audio data that can be viewed on a screen by an examiner. In one aspect, a display device comprising a computer monitor or television monitor can be used to view the video and audio data generated by the device. Depending upon the application, it may be desirable to view concurrently the video output data and the audio output data. For example, with the use of a multiplexer and quad splitter, it is possible to display on one screen the thermal image of the subject's face, the video of the subject (face and/or body), and graphical representation of the frequency and amplitude of the subject's voice. In one aspect, the display device is divided into four sections or quadrants, where during the questioning of the subject, the examiner can monitor simultaneously the thermal image of the subject's face (section 1), the video of the subject's face (section 2), the video of the entire subject (section 3), and the frequency and amplitude of the subject's voice (section 4) while the subject is being interrogated. Additional sections or quadrants are contemplated depending upon the number of thermal imagers and video cameras that are selected.

In one aspect, described herein is a stress detection device, comprising

(a) a thermal imager for detecting changes in skin temperature in a subject during the questioning of the subject, wherein the thermal imager produces a thermal analog representation, a thermal digital representation, or a combination thereof of the subject;

(b) a video camera for observing the subject during the questioning of the subject, wherein the video camera produces a video analog representation, a video digital representation, or a combination thereof of the subject;

(c) a microphone connected to a computer, wherein the computer comprises software for analyzing changes in voice frequency, voice amplitude, or a combination thereof to produce audio input data, wherein the audio input data provides a reference point for the video output data;

(d) a multiplexer, quad splitter, or a combination thereof to convert the representations produced by the thermal imager and video camera and the audio input data to video output data, wherein the video output data can be displayed; and

(e) a display device for displaying the video output data.

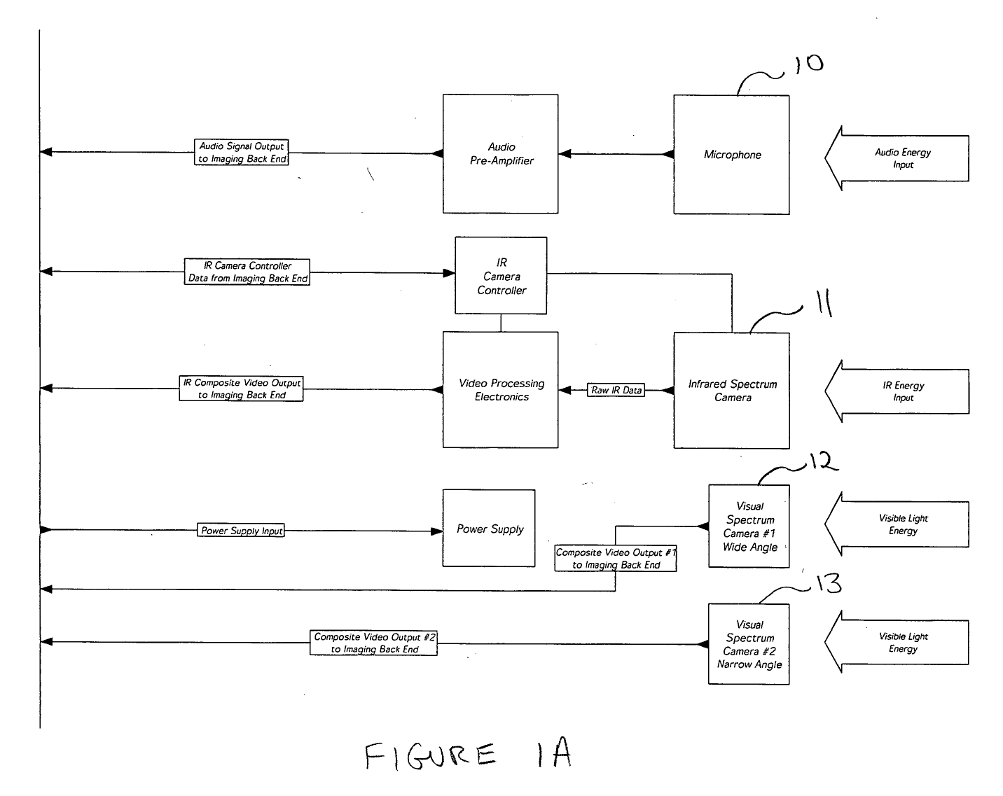

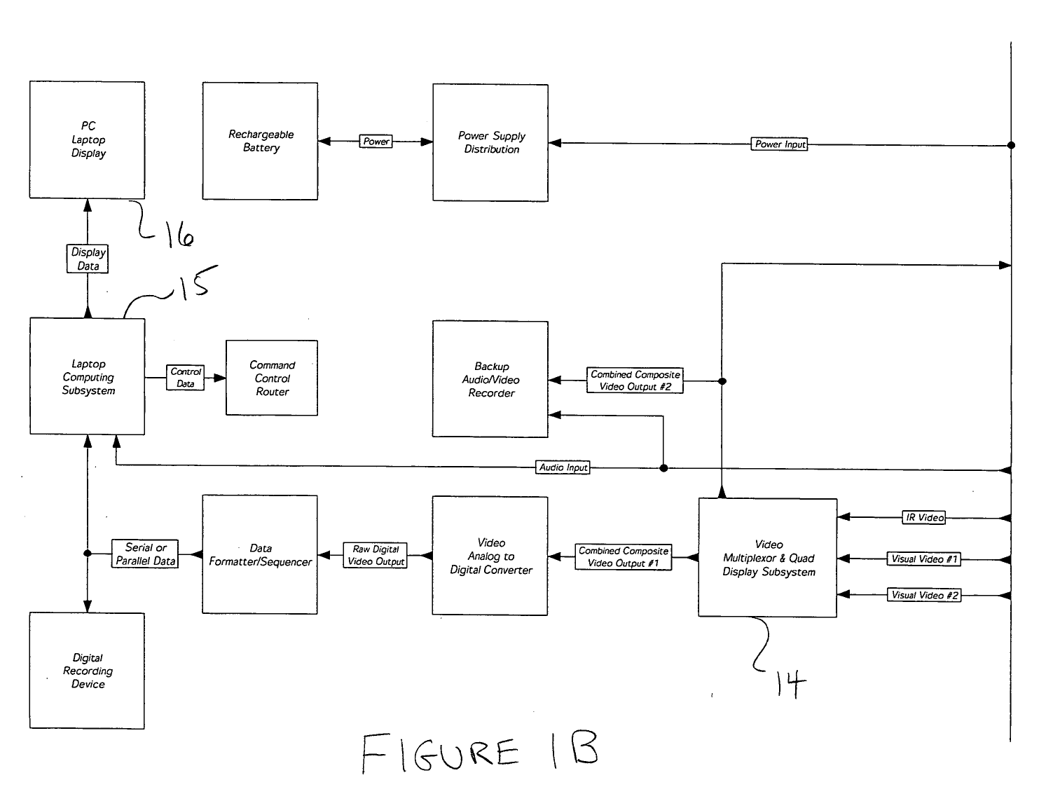

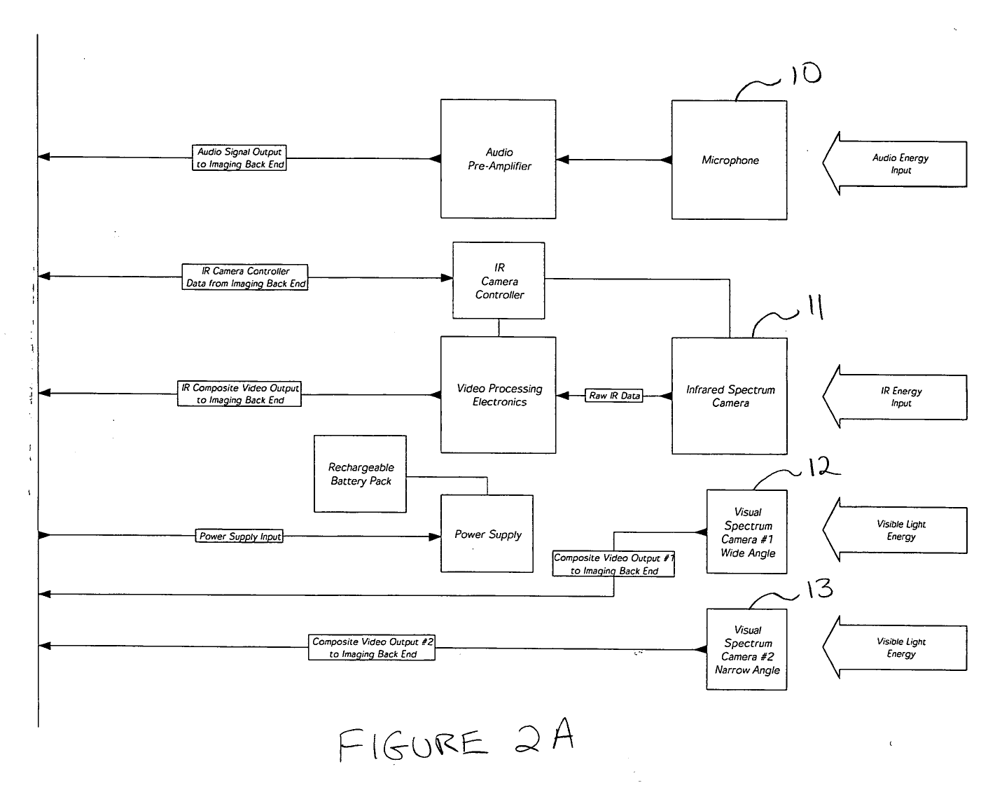

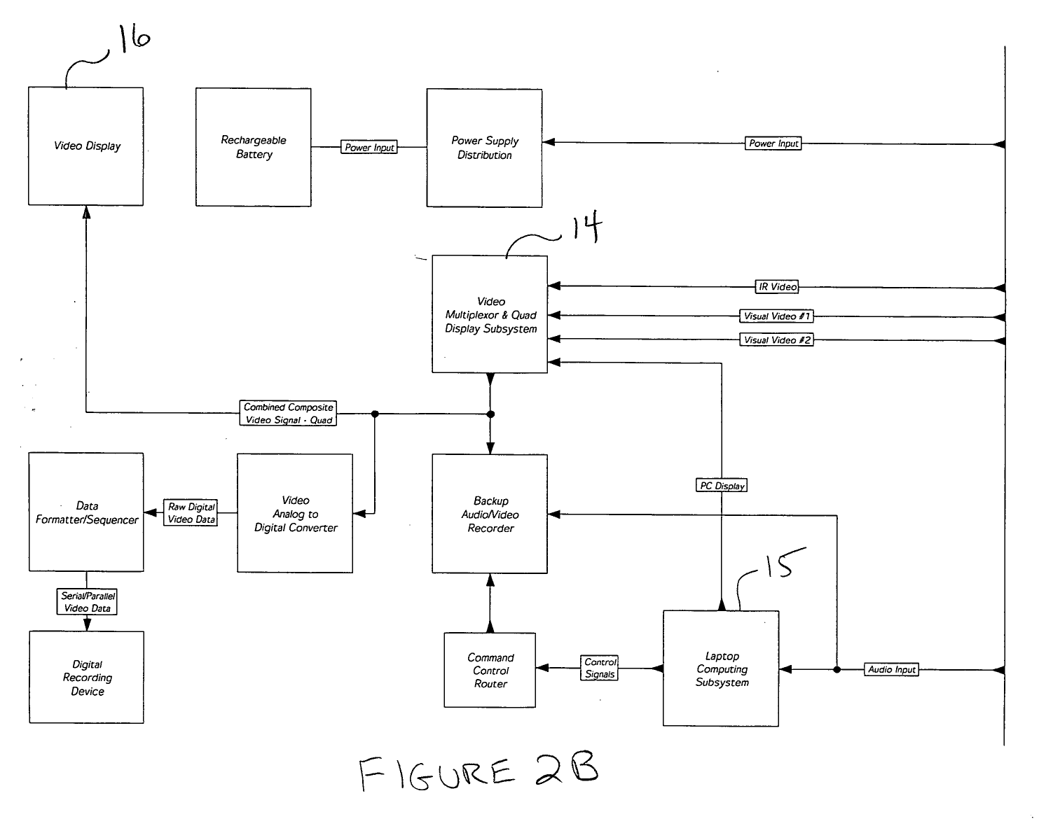

FIGS. 1 and 2 provide schematics for linking the different components of the stress detection device. Referring to FIG. 1A, the stress detection device is composed of microphone 10, an infrared spectrum camera 11, a wide-angle camera 12, and a narrow-angle camera 13. In this aspect, the infrared camera and two video cameras produce analog and/or digital representations of the subject, which is fed to a multiplexer/quad splitter 14 (FIG. 1B) to produce a single video output. The single video output can be further processed prior to being fed to a computing system 15. In FIG. 1B, the audio input data generated from the microphone 10 is fed directly to the computing system 15; however, it is contemplated that the audio input can be fed into the multiplexer/quad splitter 14 as well. The computing system 15 contains software that can convert the audio input data to a graphic representation of frequency and amplitude. The computing system will generally have a monitor for visualizing the infrared image of the subject as well as the video of the subject and changes in voice frequency and amplitude. However, it is also contemplated that the computer system can be linked to one or more monitors (e.g., television monitors).

FIG. 2 shows another schematic of the stress detection device. In this aspect, the microphone 10 produces audio input data that is fed into computing system 15. The computing system 15 produces audio output data that is fed into the multiplexer/quad splitter 14. The analog and/or digital representations generated by the infrared camera 11 and video cameras 12 and 13 is also fed into the multiplexer/quad splitter 14 to produce a single video output. The single video output can then be displayed on a monitor 16.

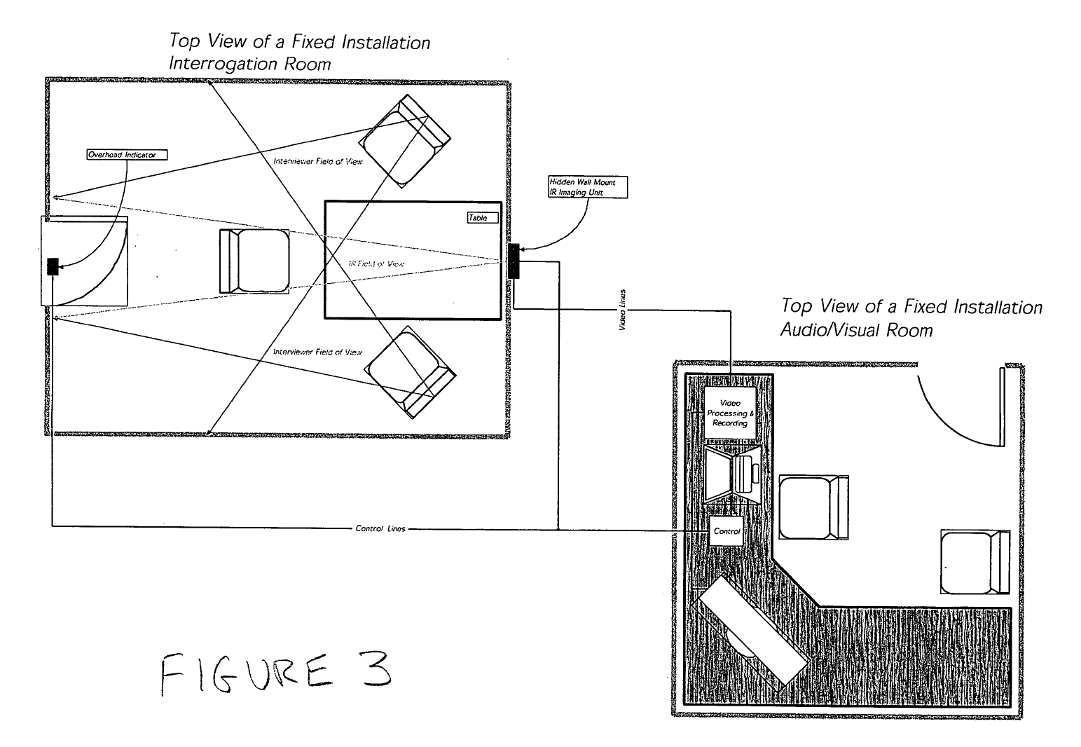

As depicted in FIGS. 1 and 2, the stress detection device can contain additional components. For example, the device can include data formatter/sequencers, digital recording devices, video analog to digital converters, backup audio/video recorders, and power supplies. The selection of components used to produce the stress detection device can vary depending upon the application of the device. In the case when the device is used in an interrogation room, infrared and video cameras can be positioned throughout the room. In one aspect, the infrared camera can be mounted on or in the wall facing the subject, where the camera hidden from the subject. This aspect is depicted in FIG. 3. Additionally, one or more video cameras can be positioned in the room to provide video of the subject during questioning. The video cameras can also be hidden from sight so that the subject is not aware that he or she is being monitored. As depicted in FIG. 3, a separate room outside the interrogation room can have a computer system that receives the audio and video input data and processes the data to provide a visual display. The selection of the computer can vary and will depend upon, among other things, the means for interleaving or de-interleaving multiple signals (e.g., quad splitter, multiplexer), the thermal imager, the video camera, the audio system, and the like.

In one aspect, the stress detection device can be a portable unit. For example, the device can be unit that contains an infrared camera, a video camera, and a multiplexer/quad splitter. The unit can be positioned in front of the subject so that the lens of the infrared camera and video camera are at a minimum focused on the neck and facial region of the subject. The unit can have one or more video cameras (narrow- or wide-view). It is contemplated that one or more video cameras can be remotely located in the interview area and send back video data wirelessly to the examiner. A microphone can also be part of the unit or a separate component. In the case when the microphone is a separate component, the microphone can be wireless or linked to the unit by cable. A laptop computer for displaying the video signal from the unit can be used. The laptop can contain the software for converting the audio input data into visual data as well. It may also be desirable to have rechargeable batteries or solar cells to power the portable device. In certain aspects, it may be desirable to use a laptop made of a durable material such as, for example, aluminum or steel, that can be used in the field. It is also contemplated that one or more examiners can have remote access to the data generated by the stress detection device. Thus, the examiner can be offsite from where the interrogation is being conducted yet still be in direct contact with the interrogator during the interrogation.

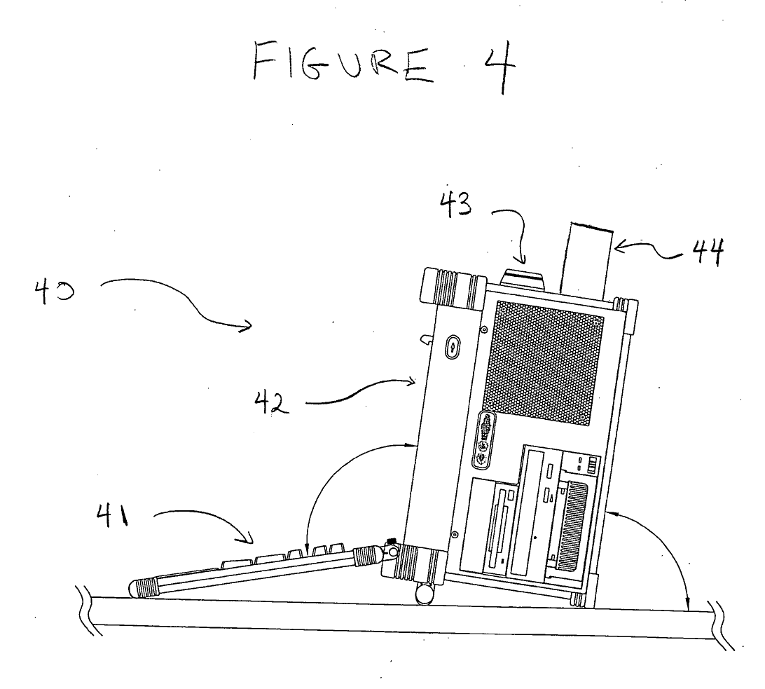

An example of a laptop useful as part of a portable stress detection device is depicted in FIG. 4. The laptop 40 is made of a durable material. The keyboard 41 folds down and can be detached and used remotely by ribbon cable or wireless, which reveals the display screen 42. An imaging box 34 is mounted to the laptop 40 near handle 43. The imaging box contains the infrared camera, at least one video camera, and a microphone. The interleaver or de-interleaver is incorporated into the laptop 40.

In general, the method for detecting stress in a subject using the devices described herein comprises:

(a) asking the subject a question in the presence of the stress detection device described herein to produce a concurrent display of change in skin temperature, audio stress level, and physical movement of the subject; and

(b) analyzing the change in skin temperature, audio stress level, and physical movement of the subject, wherein an increase in skin temperature coupled with an increase in audio stress, physical movement, or a combination thereof by the subject indicates the presence of stress in the subject.

The devices described herein have numerous applications that involve the detection of stress. The devices and methods can have applications in several areas including, but not limited to, criminology, personnel evaluation, psychiatry, clinical psychology, and self-testing for psychological behavioral assessment and feedback.

For example, the devices and methods can be used in psychiatry to evaluate levels of depression, alcoholism, drug addiction, and dementia. In addition, the devices and methods described herein can be used to assess and manage phobias.

In psychological testing, the devices and methods described herein can be used to assess the responsiveness to specific kinds of mental stress, which could be 30 important in testing of students with psychological learning disabilities, as well as of employees or potential employees for their ability to cope with stressful tasks. The identification of particular learning or work situations that are excessively stressful can be key to improving the performance of students or employees.

The devices described herein can be used to improve the training of sales personnel, negotiators, teachers or orators, all of whom must cope with mental stress. It could also measure a variety of psychological parameters, such as the types of mental tasks that the subject finds more difficult or more enjoyable. These may include reading, calculating, solving different types of problems, listening to different kinds of music, different types of jokes, different kinds of visual sceneries, etc. In such psychological applications, like in the first part of a learning disability test, the goal of the test is to identify the type of mental stimulus that evokes a change in autonomic thermoregulation larger than a given threshold.

In one aspect, the source of stress is derived from an interrogating agency such as, for example, a police interrogation. The use of lie detection devices during police interrogations has been established for some time. For example, polygraph devices have been successful in the detection of truthfulness of the suspect; however, the polygraph is not always reliable. Furthermore, the polygraph is invasive, meaning that the suspect knows he or she is being tested to determine the truthfulness of his or her statements. This can ultimately call into question the results of the polygraph test. The devices described herein are non-invasive, which means that the device is not in physical contact with the subject. However, it is contemplated that the devices described herein can be used with other invasive techniques for detecting stress such as, for example, polygraph devices or devices known in the art for detecting blood flow in the subject, measuring the respiratory rate of the subject, or detecting perspiration.

FIG. 3 depicts one aspect for using the device to detect stress. As discussed above, an infrared camera is positioned so that it can measure changes in skin temperature of the subject. The infrared camera can be pointed to any exposed skin of the subject, with the neck, face, or combination thereof as the preferred areas. One or more video cameras can be focused on the subject as well, where all of the cameras are hidden from sight from the subject. A microphone is positioned in front of the subject as well. The interrogator begins to ask the subject questions during which time video and audio input data is generated and forwarded to an audio/visual room that has a computing system and monitor. The audio and video data is converted to visual data that can be displayed on the monitor for the examiner's review. The examiner can review concurrently changes in skin temperature and voice frequency/amplitude as well as monitor the subject's movements. If there is a noticeable change in skin temperature, voice frequency/amplitude, and/or movement/behavior of the subject, the examiner can notify the interrogator that a certain line of questioning is creating more stress in the subject with the use of a feedback system. The feedback system is a way the examiner can provide comments to the interrogator regarding output produced by the stress detection device. This can be useful in aiding the interrogator on where to focus the line of questioning. For example, the examiner can notify the interrogator by a flashing light system, (e.g., green light is little to no stress or red light is an indication of stress), which is indicated in FIG. 3 as the overhead indicator. In other aspects, a variable rate indicator can be used to provide feedback to the interrogator. Alternatively, the interrogator can have an earpiece or pda (e.g., remote or wireless access) that is not obvious to the subject so that the examiner can be in direct communication with the interrogator.

The stress detection devices can produce real-time visual displays for detecting stress. However, it is contemplated that the visual displays can be stored for future use and review. In certain aspects, if a certain skin temperature is reached, the examiner will manually take one or more snapshots of the subject (e.g., skin temperature, audio frequency/amplitude) for future review. Here, the skin temperature of the subject can “trigger” the examiner to take a snapshot. In the alternative, the device can be configured so that snapshots of the subject are taken at specified time intervals. The snapshots are useful for comparing the subjects stress levels over certain periods of time during questioning. If desired, the snapshots can be displayed in the monitor so that they can be viewed concurrently while viewing thermal, video, and audio images generated by the subject. The “snapshots” are also referred to as a thumb-nail. When thumb-nails are used, the examiner can scan the monitor and pick out the thermal spikes, then review the corresponding video at a particular time stamp and refer back to the audio at the same point in time and listen to the conversation that generated the thermal spike. It is also contemplated that the thermal, video, and audio images generated by the device can be viewed simultaneously on one display device or, in the alternative, any combination of thermal, video, and audio images can be displayed.

The devices and methods described herein provide advantages over previous techniques. With respect to the audio and video data, the devices described herein provide a reference point between audio output data and the video output data. This is important with respect to correlating the audio data with the thermal and video data. The devices described herein use more than just a microphone in combination with the thermal imager and video camera. The audio data produced by the subject is converted to a graphical display of frequency and amplitude so that a change in audio frequency and amplitude can be correlated with a change in skin temperature and/or body movements of the subject. In other words, the audio information can be displayed in the time or frequency domain allowing visual comparison to the thermal and video data being generated during the interview. This feature is not appreciated by the art and provides a more reliable way to detect stress in a subject.

Throughout this application, various publications are referenced. The disclosures of these publications in their entireties are hereby incorporated by reference into this application in order to more fully describe the compounds, compositions and methods described herein.

Various modifications and variations can be made to the materials, methods, and articles described herein. Other aspects of the devices and methods described herein will be apparent from consideration of the specification and practice of the materials, methods, and articles disclosed herein. It is intended that the specification and examples be considered as exemplary.

What is claimed is:

1. A stress detection device, comprising
(a) a thermal imager for detecting changes in skin temperature in a subject during the questioning of the subject, wherein the thermal imager produces a thermal analog representation, a thermal digital representation, or a combination thereof of the subject;
(b) a video camera for observing the subject during the questioning of the subject, wherein the video camera produces a video analog representation, a video digital representation, or a combination thereof of the subject;
(c) a means for interleaving or de-interleaving multiple representations from (a) and (b);
(d) a means for providing a reference point between audio output data and the representations from (a) and (b); an
(e) a display device for displaying the representations and the audio output data.

2. The device of claim 1, wherein the thermal imager comprises an infrared camera.

3. The device of claim 1, wherein the means for providing a reference point between audio output data and the thermal analog and/or digital representations and video analog and/or digital representations comprises a microphone connected to a computer comprising software for analyzing changes in voice frequency, voice amplitude, or a combination thereof.

4. The device of claim 1, wherein the device further comprises a means for detecting changes in audio stress level of the subject during the questioning of the subject.

5. The device of claim 1, wherein the video camera comprises at least one CCD camera.

6. The device of claim 1, wherein the video camera comprises two CCD cameras.

7. The device of claim 1, wherein the means for interleaving or de-interleaving the thermal analog and/or digital representations and the video analog and/or digital representations to video output data comprises a multiplexer.

8. The device of claim 1, wherein the means for interleaving or de-interleaving the thermal analog and/or digital representations and the video analog and/or digital representations to video output data comprises a quad splitter.

9. The device of claim 1, wherein the means for interleaving or de-interleaving the thermal analog and/or digital representations and the video analog and/or digital representations to video output data comprises a combination of a multiplexer and a quad splitter.

10. The device of claim 1, wherein the means for interleaving or de-interleaving the thermal analog and/or digital representations and the video analog and/or digital representations to video output data comprises a multiplexer, wherein the multiplexer can further interleave or de-interleave the audio output data.

11. The device of claim 1, wherein the means for interleaving or de-interleaving the thermal analog and/or digital representations and the video analog and/or digital representations to video output data comprises a quad splitter, wherein the quad splitter can further interleave or de-interleave the audio output data.

12. The device of claim 1, wherein the means for interleaving or de-interleaving the thermal analog and/or digital representations and the video analog and/or digital representations to video output data comprises a combination of a multiplexer and a quad splitter, wherein the multiplexer and quad splitter can further interleave or de-interleave the audio output data.

13. The device of claim 1, wherein the display device comprises a computer monitor or television monitor.

14. The device of claim 1, wherein the display device concurrently displays the video output data and the audio output data.

15. The device of claim 1, wherein the device is non-invasive.

16. The device of claim 1, wherein the device is portable.

17. The device of claim 1, wherein the device is non-portable.

18. The device of claim 1, wherein the device further comprises a means for recording audio output, video input, and thermal input.

19. The device of claim 1, wherein the device further comprises a feedback system.

20. A stress detection device, comprising
(a) a thermal imager for detecting changes in skin temperature in a subject during the questioning of the subject, wherein the thermal imager produces a thermal analog representation, a thermal digital representation, or a combination thereof of the subject;
(b) a video camera for observing the subject during the questioning of the subject, wherein the video camera produces a video analog representation, a video digital representation, or a combination thereof of the subject;
(c) a microphone connected to a computer, wherein the computer comprises software for analyzing changes in voice frequency, voice amplitude, or a combination thereof to produce audio input data, wherein the audio input data provides a reference point for the video output data;
(d) a multiplexer, quad splitter, or a combination thereof to convert the representations produced by the thermal imager and video camera, and the audio input data to video output data, wherein the video output data can be displayed; and
(e) a display device for displaying the video output data.

21. A method for detecting stress in a subject, comprising
(a) asking the subject a question in the presence of the device of claim 1 to produce a concurrent display of change in skin temperature, audio stress level, and physical movement of the subject; and
(b) analyzing the change in skin temperature, audio stress level, and physical movement of the subject, wherein an increase in skin temperature coupled with an increase in audio stress, physical movement, or a combination thereof by the subject indicates the presence of stress in the subject.

22. The method of claim 21, wherein the thermal imager is pointed at the neck, face, or combination thereof of the subject.

23. The method of claim 21, wherein the video camera comprises two CCD cameras, wherein the first CCD camera is pointed at the neck, face, or combination thereof of the subject and the second CCD camera is pointed at the entire body of the subject.

24. The method of claim 21, wherein the source of stress is derived from an interrogating agency.

25. The method of claim 21, wherein the method comprises an interrogator and an examiner, wherein upon questioning of the subject by the interrogator, the examiner reviews the change in skin temperature, audio stress level, and physical movement of the subject, wherein the examiner indicates to the interrogator when the subject is undergoing stress during questioning of the subject.

26. A method for detecting stress in a subject, comprising
(a) asking the subject a question in the presence of the device of claim 20 to produce a concurrent display of change in skin temperature, audio stress level, and physical movement of the subject; and
(b) analyzing the change in skin temperature, audio stress level, and physical movement of the subject, wherein an increase in skin temperature coupled with an increase in audio stress, physical movement, or a combination thereof by the subject indicates the presence of stress in the subject.
